(19) United States
(12) Patent Application Publication
(10) Pub. No.: US 2013/0079652 A1
(43) Pub. Date: Mar. 28, 2013

(54) ASSESSMENT OF CARDIAC HEALTH BASED ON HEART RATE VARIABILITY

(71) Applicant: Elyasaf Korenweitz, Elon-Moreh (IL)

(72) Inventor: Elyasaf Korenweitz, Elon-Moreh (IL)

(73) Assignee: VITALCARE MEDICAL LTD., Jerusalem (IL)

(21) Appl. No.: 13/636,129

(22) PCT Filed: Mar. 17, 2011
(86) PCT No.: PCT/IB2011/051114

§ 371 Date: Oct. 18, 2012

Related U.S. Application Data
(60) Provisional application No. 61/315,958, filed on Mar. 21, 2010.

(51) Int. Cl. A61B 5/024; A61B 5/0432; A61B 5/0456; A61B 5/0452; A61B 5/04

(52) U.S. Cl. CPC A61B 5/02405; A61B 5/0452; A61B 5/04012; A61B 5/0456; A61B 5/0432
USPC 600/515

(57) ABSTRACT

A diagnostic method includes receiving data comprising a series of heartbeat intervals acquired from a patient (22). A first type of computation, selected from a group of computation types consisting of time-domain analysis (82), frequency-domain analysis (84), and nonlinear fractal analysis (86), is applied to the data in order to compute a first measure of heart rate variability (HRV) of the patient. A second type of computation, selected from the group and different from the first type, is applied to the data in order to compute a second measure of the HRV of the patient. At least the first and second measures are combined so as to derive a parameter indicative of a condition of cardiac health of the patient.

### CROSS-REFERENCE TO RELATED APPLICATION

This application claims the benefit of U.S. Provisional Patent Application 61/315,958, filed Mar. 21, 2010. This application is a continuation-in-part of PCT Patent Application PCT/IL2009/001189, filed Dec. 15, 2009, which claims the benefit of U.S. Provisional Patent Application 61/193,674, filed Dec. 15, 2008. All of these related applications are incorporated herein by reference.

### FIELD OF THE INVENTION

The present invention relates generally to medical diagnostic systems and methods, and particularly to assessment of cardiovascular health.

### BACKGROUND OF THE INVENTION

Analysis of variability in heart rate has been used to assess physiological function. The variability is measured over a sequence of inter-beat intervals, which may be conveniently measured by analyzing physiological signals, such as the electrocardiogram (ECG). Most commonly, the inter-beat intervals are measured based on the time span between R wave peaks (RR intervals) in the electrocardiogram signal.

Various signal analysis and processing techniques have been applied to heart rate variability (HRV) data. Some of these techniques are based on time-domain analysis and may use Poincaré plots, for example. A Poincaré plot is a scatter plot in which each RR interval is plotted against the preceding RR interval, so that the (x,y) coordinates of each point in the plot are (RRᵢ, RRᵢ₊₁). A method for HRV analysis based on Poincaré plots is described, for example, in U.S. Pat. No. 6,731,974.

Other HRV analysis techniques use frequency-domain analysis. For example, U.S. Pat. No. 6,532,382 describes a method of calculating the HRV on the basis of the Fourier transform, wherein the frequency spectrum of certain measuring values is calculated from the Fourier coefficients.

Still other HRV analysis techniques use nonlinear fractal analysis. Such methods are described, for example, by Tan et al., in “Fractal Properties of Human Heart Period Variability: Physiological and Methodological Implications,” Journal of Physiology 587:15 (Aug. 1, 2009), pages 3929-3941, which is incorporated herein by reference. The authors use fractal frequency scaling of heart period variability as a concise index of overall cardiac control. For each subject, the fractal scaling component was estimated by detrended fluctuation analysis (DFA). Other fractal-based methods are described by Porta et al., in “Multimodal Signal Processing for the Analysis of Cardiovascular Variability,” Philosophical Transactions of the Royal Society A 367 (Jan. 28, 2009), pages 391-409, which is also incorporated herein by reference.

### SUMMARY

Embodiments of the present invention that are described hereinbelow provide methods, systems and software for analysis of HRV.

There is therefore provided, in accordance with an embodiment of the present invention, a diagnostic method, which includes receiving data including a series of heartbeat intervals acquired from a patient. A first type of computation, selected from a group of computation types consisting of time-domain analysis, frequency-domain analysis, and nonlinear fractal analysis, is applied to the data in order to compute a first measure of heart rate variability (HRV) of the patient. A second type of computation, selected from the group and different from the first type, is applied to the data in order to compute a second measure of the HRV of the patient. At least the first and second measures are combined so as to derive a parameter indicative of a condition of cardiac health of the patient.

Combining at least the first and second measures may include computing a weighted sum of the first and second measures and/or a non-linear function of at least one of the first and second measures, such as raising at least one of the first and second measures to a power not equal to one.

Typically, the method includes setting a threshold, and comparing the parameter to the threshold in order to provide a prognostic classification of the patient.

In disclosed embodiments, the time-domain analysis includes computing a variation of the heartbeat intervals between pairs of the heartbeats as a function of a lag between the heartbeats in each pair, and deriving a measure from the function. The time-domain analysis typically includes computing a measure selected from a set of measures consisting of a mean RR interval; a standard deviation of RR intervals; a ratio of standard deviations along different axes in a scatter plot of the RR intervals; a difference between ratios of standard deviations along different axes in scatter plots of the RR intervals computed for different lags between heartbeats; a standard deviation of an average of RR intervals in different time segments; a mean square difference between adjacent RR intervals; and a fraction of differences between adjacent RR intervals that are greater than a certain time threshold.

The frequency-domain analysis typically includes computing a measure selected from a set of measures consisting of a total spectral power of all RR intervals up to a frequency cutoff; a total spectral power of all RR intervals in a specified frequency range; and a ratio of power components of the RR intervals in two different frequency ranges.

The nonlinear fractal analysis typically includes computing a measure selected from a set of measures consisting of a self-similar fractal scaling; a log transformation of a head of a detrended fluctuation analysis (DFA) graph; and a log transformation of a tail of a detrended fluctuation analysis (DFA) graph.

There is also provided, in accordance with an embodiment of the present invention, a diagnostic method, which includes receiving data including a series of heartbeat intervals acquired from a patient. A variation of the heartbeat intervals between pairs of the heartbeats is computed as a function of a lag between the heartbeats in each pair. The computed variation is analyzed in order to assess a condition of cardiac health of the patient.

In disclosed embodiments, computing the variation includes finding a ratio of first and second deviances along respective first and second axes in a scatter plot of the pairs, and analyzing the computed variation includes fitting a parametric curve to the function, and comparing at least one parameter of the curve to a predefined criterion in order to assess the condition. In one embodiment, fitting the parametric curve includes fitting a quadratic logarithmic function, and wherein comparing the at least one parameter includes checking a sign of a parameter that multiplies a linear term in the function in order to assess the cardiac health.

There is additionally provided, in accordance with an embodiment of the present invention, diagnostic apparatus, including a memory, which is configured to receive data including a series of heartbeat intervals acquired from a patient. A processor is configured to apply a first type of computation, selected from a group of computation types consisting of time-domain analysis, frequency-domain analysis, and nonlinear fractal analysis, to the data in order to compute a first measure of heart rate variability (HRV) of the patient, to apply a second type of computation, selected from the group and different from the first type, to the data in order to compute a second measure of the HRV of the patient, and to combine at least the first and second measures so as to derive a parameter indicative of a condition of cardiac health of the patient.

There is further provided, in accordance with an embodiment of the present invention, diagnostic apparatus, including a memory, which is configured to receive data including a series of heartbeat intervals acquired from a patient. A processor is configured to compute a variation of the heartbeat intervals between pairs of the heartbeats as a function of a lag between the heartbeats in each pair, and to analyze the computed variation in order to assess a condition of cardiac health of the patient.

There is moreover provided, in accordance with an embodiment of the present invention, a computer software product, including a computer-readable medium in which program instructions are stored, which instructions, when read by a processor, cause the processor to receive data including a series of heartbeat intervals acquired from a patient, to apply a first type of computation, selected from a group of computation types consisting of time-domain analysis, frequency-domain analysis, and nonlinear fractal analysis, to the data in order to compute a first measure of heart rate variability (HRV) of the patient, to apply a second type of computation, selected from the group and different from the first type, to the data in order to compute a second measure of the HRV of the patient, and to combine at least the first and second measures so as to derive a parameter indicative of a condition of cardiac health of the patient.

There is furthermore provided, in accordance with an embodiment of the present invention, a computer software product, including a computer-readable medium in which program instructions are stored, which instructions, when read by a processor, cause the processor to receive data including a series of heartbeat intervals acquired from a patient, to compute a variation of the heartbeat intervals between pairs of the heartbeats as a function of a lag between the heartbeats in each pair, and to analyze the computed variation in order to assess a condition of cardiac health of the patient.

The present invention will be more fully understood from the following detailed description of the embodiments thereof, taken together with the drawings in which:

### BRIEF DESCRIPTION OF THE DRAWINGS

FIG. 1 is a block diagram that schematically illustrates a system for assessment of cardiac health, in accordance with an embodiment of the present invention;

FIG. 2 is a lag plot of HRV, in accordance with an embodiment of the present invention;

FIG. 3 is a flow chart that schematically illustrates a method for analyzing HRV, in accordance with an embodiment of the present invention;

FIGS. 4A-4J are plots showing a standard deviation ratio of HRV as a function of lag for ten healthy patients, in accordance with an embodiment of the present invention;

FIGS. 5A-5J are plots showing a standard deviation ratio of HRV as a function of lag for ten patients suffering from heart disease, in accordance with an embodiment of the present invention; and

FIG. 6 is a flow chart that schematically illustrates a method for deriving a measure of cardiac health, in accordance with an embodiment of the present invention.

### DETAILED DESCRIPTION OF EMBODIMENTS

### Overview

HRV has been widely studied by scientists, and differences in HRV among different patient groups have been correlated with cardiovascular disease. There remains a need, however, for new methods of HRV analysis that can provide a simple, unequivocal prognostic indicator. Embodiments of the present invention that are described here in below address this need by providing methods for processing HRV data, which comprises a series of heartbeat intervals acquired from a patient, in order to derive one or more parameters indicative of the patient's condition of cardiac health. The inventors have found that these parameters can be compared to certain predefined criteria (such as thresholds), in order to give a prognostic assessment with high sensitivity and specificity.

Some of the disclosed embodiments analyze HRV by combining at least two different types of computations. The different types may include time-domain analysis, frequency-domain analysis, and/or nonlinear fractal analysis. Each type gives its own measure of HRV, and these different measures are combined in order to give the desired cardiac health parameter. The combination may be linear, such as a weighted sum of the measures, or it may involve non-linear functions, such as raising at least one of the measures to a power not equal to one.

In some embodiments, the variation of the heartbeat intervals between pairs of the heartbeats is computed as a function of a lag between the heartbeats in each pair. This functional characteristic of the HRV has been found to be a good indicator of cardiac health in and of itself. Alternatively or additionally, this lag-based analysis may be used as one of the types of computation that are combined to give a cardiac health parameter, as described above.

The variation of the heartbeat intervals as a function of the lag may be computed as a ratio of deviances measured along different axes in a scatter plot (such as a Poincaré plot) of the pairs of heartbeat intervals. In the above-mentioned PCT patent application, the difference between these deviance ratios for two specified values of the lag is used as the cardiac health parameter, referred to by the inventors as “adaptation.” In another embodiment, which is described further hereinbelow, a parametric curve is fitted to the functional variation of the deviance ratio with lag, and the cardiac health parameter is derived from one or more parameters of the curve.

### System Description

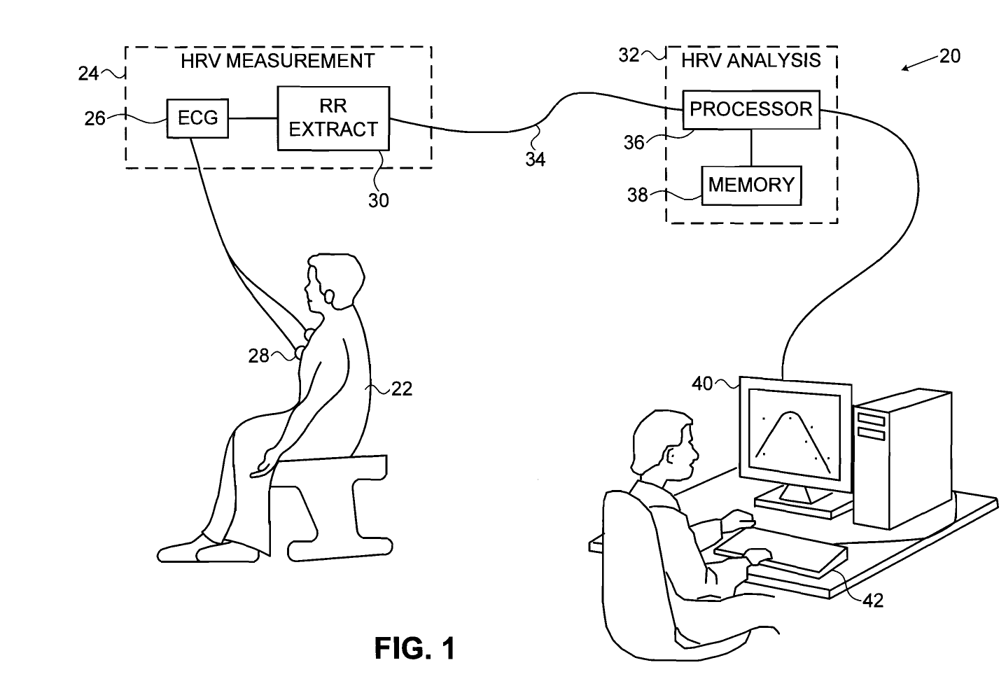

FIG. 1 is a block diagram that schematically illustrates a system 20 for assessing the cardiac health of a patient 22, in accordance with an embodiment of the present invention. A HRV measurement module 24 comprises ECG input circuitry 26, which receives and processes ECG signals from electrodes 28 that are fixed to the body of patient 22. A RR extraction circuit 30 processes the ECG signals in order to identify the QRS complex and measure the intervals between successive R-peaks. These functions of module 24 are standard features of many commercially-available ECG measurement and monitoring systems.

Module 24 outputs the series of heartbeat intervals acquired from patient 22 to a HRV analysis module 32 via a communication link 34 (which may be wired or wireless, or a combination of the two). Module 32 may be located remotely from module 24, in which case link 34 may comprise parts of a local and/or wide area network. In this configuration, analysis module 32 may receive data, either on-line or prerecorded, from multiple different HRV measurement modules in different locations. Alternatively, modules 24 and 32 may be collocated, and may even be contained in a common enclosure as parts of a single measurement and analysis unit (in which case link 34 may simply comprise an internal connection within the unit). All of these alternative configurations are considered to be within the scope of the present invention.

HRV analysis module 32 typically comprises a general-purpose computer, comprising a programmable processor 36 and a memory 38, which receives and stores the data from module 24. Processor 36 is typically programmed in software to carry out the HRV analysis functions that are described herein. This software may be downloaded to module 32 in electronic form, over a network, for example. Alternatively or additionally, the software may be stored on tangible, computer-readable storage media, such as optical, magnetic, or electronic memory media. Further alternatively or additionally, at least some of the functions of processor 36 may be carried out by signal processing and logic hardware components, which may be hard-wired or programmable.

Typically, processor 36 outputs the analysis results to a display 40. The results typically comprise a diagnostic or prognostic parameter, indicative of the condition of the patient's cardiac health. Additionally or alternatively, display 40 may present the results of analysis in more complex numerical and/or graphical form. A user of system 20, such as a physician, may operate a user interface 42 to query and control the operation of module 32.

### Lag-Based Analysis

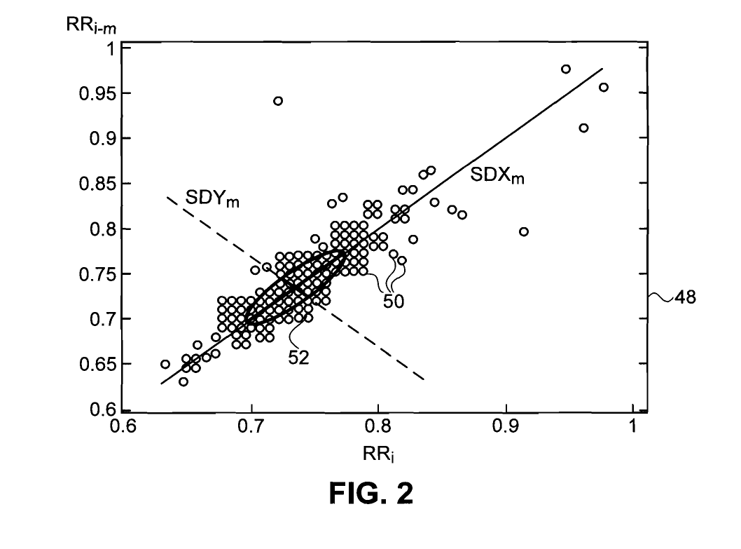

FIG. 2 is a lag plot 48 of HRV, in accordance with an embodiment of the present invention. Plot 48 has the form of a scatter plot, similar to a Poincaré plot, except that data points 50 in plot 48 correspond to pairs of heartbeat intervals that are separated by a lag of m heartbeats. In other words, each data point 50 has coordinates (RRᵢ, RRᵢ₋ₘ). HRV analysis module 32 creates and analyzes plots of this sort for multiple different values of m. Points 50 in plot 48 have a vectorial deviance that is represented by an oval 52. Typically, for small values of m, for which the heartbeats in the pair are highly correlated, oval 52 has the elongated form that is shown in FIG. 2, and the oval grows increasingly rounder as m increases and the correlation between the heartbeats in each pair drops.

To analyze plot 48, processor 36 rotates the axes by 45°, defining a new (x,y) coordinate system defined by the relations:

$${x}_{m i} = \frac{\sqrt{2}}{2} {RR}_{i} + \frac{\sqrt{2}}{2} {RR}_{i - m} \\ {y}_{m i} = - \frac{\sqrt{2}}{2} {RR}_{i} + \frac{\sqrt{2}}{2} {RR}_{i - m} \qquad ( 1 )$$

These axes are shown as dashed lines in plot 48, with their origin at the mean of the distribution of data points 50. Processor 36 computes a measure of the deviance of the data points along each of these axes, such as the statistical standard deviations, SDXₘ and SDYₘ, for each of the analyzed values of m.

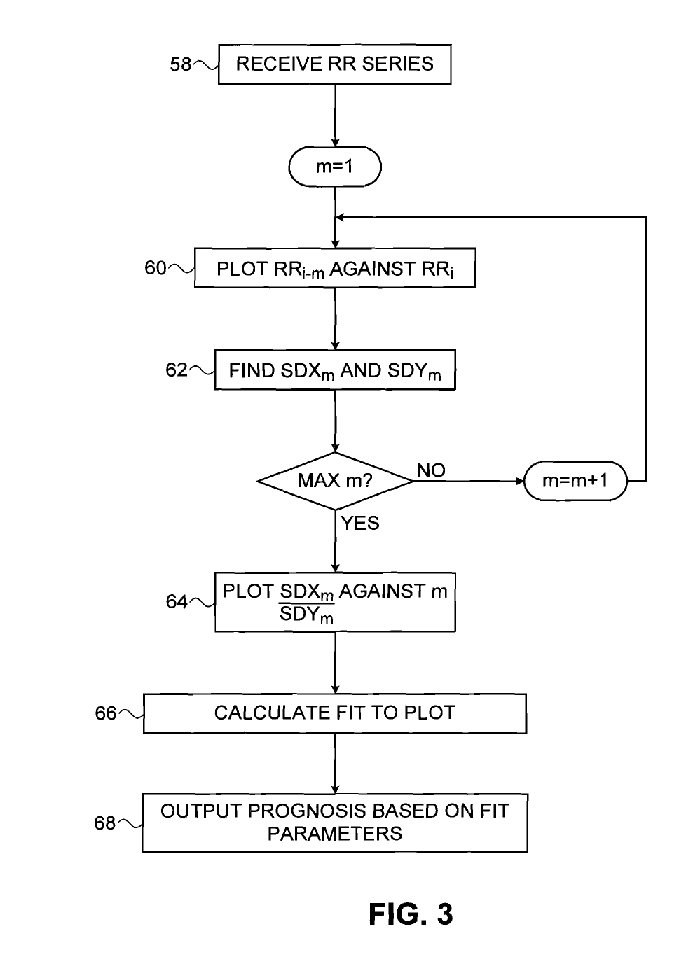

FIG. 3 is a flow chart that schematically illustrates a method applied by analysis module 32 for lag-based analysis of HRV, in accordance with an embodiment of the present invention. The analysis module applies the method to data comprising series of heartbeat intervals (typically RR intervals) that it receives from measurement module 24, at a data input step 58. The data may be filtered to remove spurious data points, as described, for example, in the above-mentioned PCT Patent Application PCT/IL2009/001189.

For each of the values of the lag m that is to be used in the analysis, processor 36 plots RRᵢ₋ₘ against RRᵢ, at a scatter plotting step 60. The term “plotting” in the present context does not necessarily mean actually creating a graphical representation of the data as shown in FIG. 2, but rather may simply comprise placing the data in a suitable data structure to facilitate the subsequent analysis, and specifically computation of the X- and Y-deviances, as defined above. Typically, processor 36 performs the analysis for each value of m from one up to a predetermined maximum lag value, which is typically on the order of ten.

For each value of m, processor 36 computes the standard deviation values SDXₘ and SDYₘ, at a deviance computation step 62. The processor then plots the deviance ratio

$$r = \frac{{SDX}_{m}}{{SDY}_{m}}$$

as a function of m, at a deviance plotting step 64 (wherein “plot” is again used in the broad sense defined above). The functional dependence of the deviance ratio on m typically exhibits a decay with large m, due to the decreasing correlation between beats as lag increases; but the form of the dependence at low and intermediate m is a strong indicator of cardiac health. To extract this dependence, processor 36 fits a parametric curve to the plot of r against m, at a fitting step 66.

Various types of curves may be used at step 66, but the inventors have found a parametric quadratic logarithmic curve, such as the following, to give useful results:)

r=a+b ln m+c(ln m)² (2)

Here a, b and c are constant parameters, whose values are adjusted by least-squares fitting to the data. Alternatively, other parametric functional forms and fitting methods may be used, as are known in the art. Processor 36 compares one or more of the resulting fit parameters to a predefined criterion, such as a threshold, in order to give an indication of the patient's condition, at an output step 68. The inventors have found that the sign of the factor b, which multiplies the linear term in equation (2), is a strong indicator of cardiac prognosis: Negative values of b are characteristic of healthy hearts and correlate with favorable prognosis, while positive values are indicative of heart disease and a high likelihood of ensuing complications.

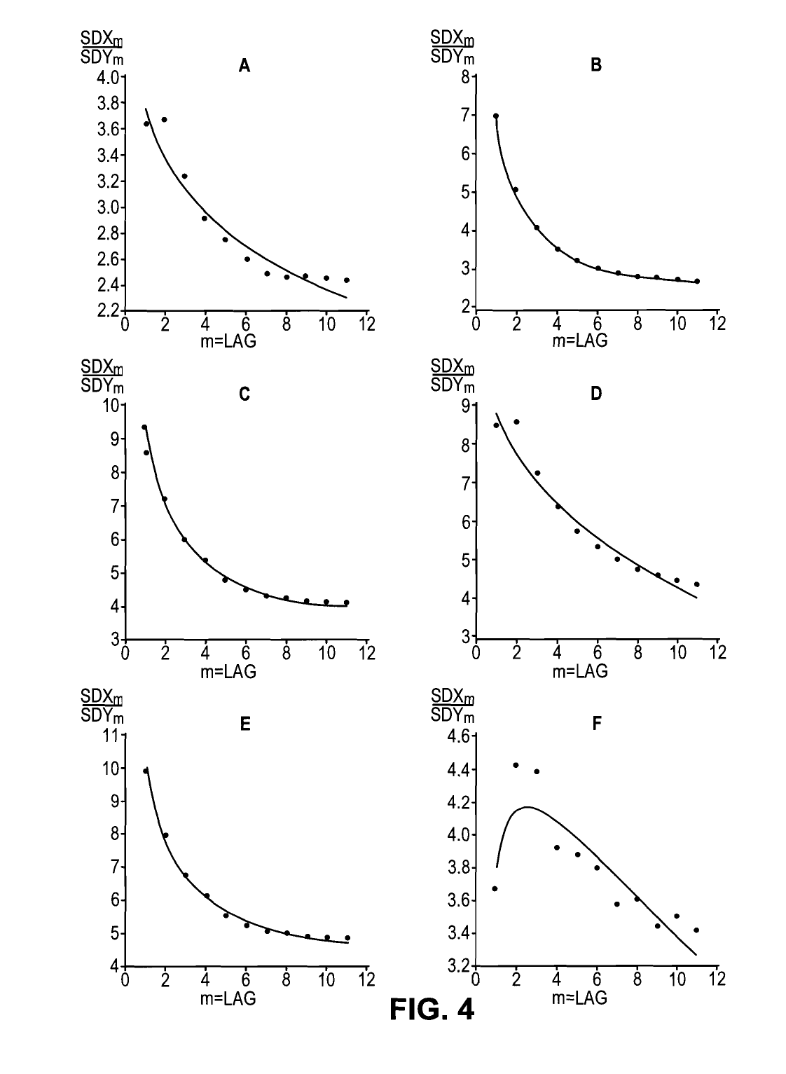

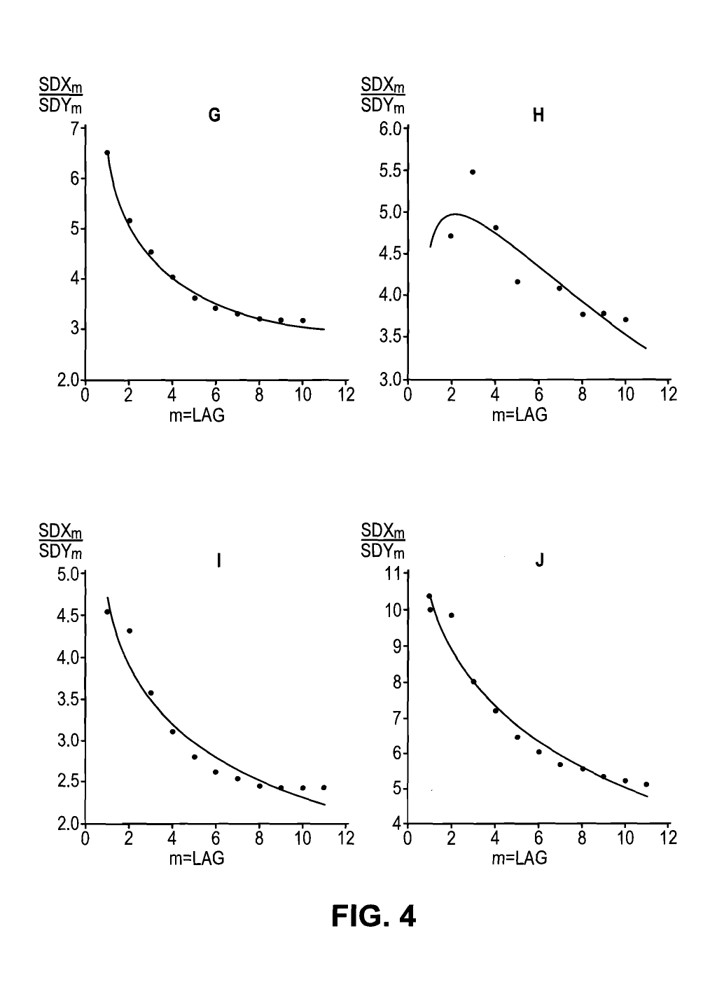

FIGS. 4A-4J are plots showing the deviance ratio

$$r = \frac{{SDX}_{m}}{{SDY}_{m}}$$

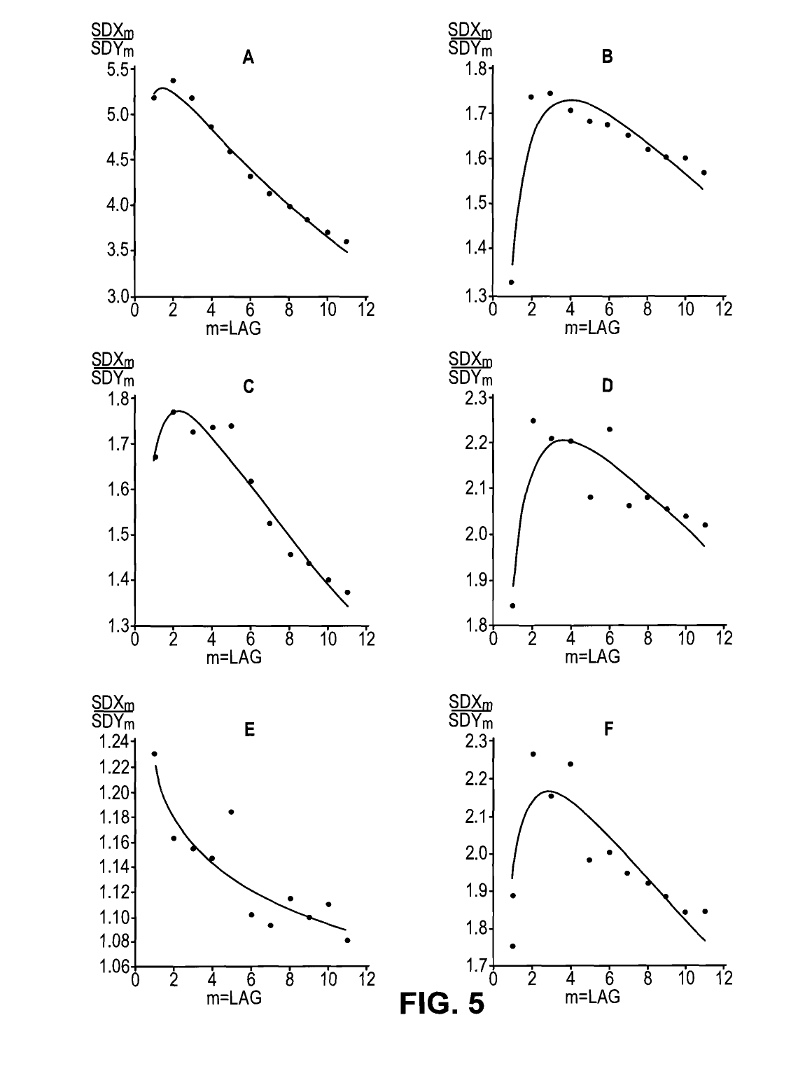

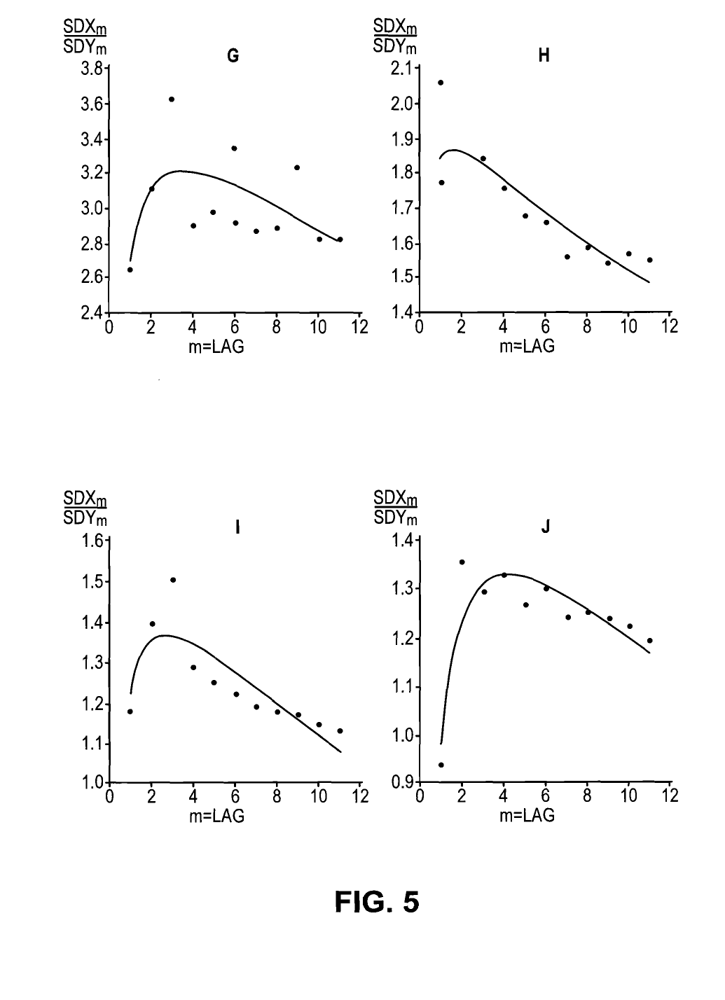

as a function of the lag m for ten healthy patients, while FIGS. 5A-5J are plots showing r as a function of m for ten patients suffering from heart disease, in accordance with an embodiment of the present invention. The HRV data used in the analysis were taken from the RR-Interval Substudy Database of the Cardiac Arrhythmia Suppression Trial (CAST). The healthy and diseased patients whose RR-interval data were used in generating the present plots were selected at random from the set of 734 patients in the Substudy Database.

Each of FIGS. 4A-4J and 5A-5J contains data points corresponding to the value of r for each value of m and a curve corresponding to the parametric fit of equation (2) to the data points. The fit parameters are shown in the table below:

TABLE I
FITTING PARAMETERS

| FIG. | a | b | c |
|---|---|---|---|
| 4A | 5.2155 | 0.3452 | −0.4439 |
| 4B | 1.3701 | 0.5219 | −0.1890 |
| 4C | 1.6619 | 0.2729 | −0.1692 |
| 4D | 1.8855 | 0.4933 | −0.1898 |
| 4E | 1.2202 | −0.5080 | 0.0015 |
| 4F | 1.9345 | 0.4448 | −0.2143 |
| 4G | 2.6959 | 0.8165 | −0.3215 |
| 4H | 1.8420 | 0.0955 | −0.1025 |
| 4I | 1.2243 | 0.2843 | −0.1433 |
| 4J | 0.9828 | 0.4870 | −0.1709 |
| 5A | 3.7525 | −0.5214 | −0.0354 |
| 5B | 6.9587 | −3.3202 | 0.6542 |
| 5C | 9.3746 | −4.0257 | 0.7380 |
| 5D | 8.7503 | −1.2184 | −0.3102 |
| 5E | 9.9461 | −3.7080 | 0.6374 |
| 5F | 3.8078 | 0.7793 | −0.4195 |
| 5G | 6.5384 | −2.2930 | 0.3448 |
| 5H | 4.5910 | 0.9835 | −0.6235 |
| 5I | 4.7267 | −1.2165 | 0.0769 |
| 5J | 10.3638 | −2.0152 | −0.1307 |

It can be seen in the table above that the parameter b was positive for all but one of the healthy patients and negative for all but two of the diseased patients. Thus, b is an accurate indicator of heart condition. Alternatively, more complex combinations of the fit parameters may be used in deriving other indicators of cardiac health.

### Generalized Parameters

As noted earlier, the inventors have found that certain generalized parameters, derived from combinations of different types of measures of HRV, predict cardiac health with high sensitivity and specificity. The different types of measures include time-domain, frequency-domain, and fractal analysis measures. Some examples of each are presented below, along with abbreviations that are used in the description that follows:

### Time-Domain Measures

Mean RR interval (AVNN).
Standard deviation of the RR intervals (SDNN).
Standard deviation along the X-axis of the rotated Poincare plot (SDX, which is equal to SDXₘ for m=1).
Standard deviation along the Y-axis of the rotated Poincare plot (SDY, which is equal to SDYₘ for m=1).
The deviance ratio

$$\frac{SDX}{SDY}$$

(which is equal to r for m=1) (RATIO).
Standard deviation of the average of RR intervals in different time segments (for example, all five-minute segments of a 24-hour recording) (SDANN).
Square root of the mean square difference between adjacent RR intervals (rMSSD).
Fraction of differences between adjacent RR intervals that are greater than a certain time threshold, expressed as n milliseconds (pNNn).
Fit parameters a, b and c from equation (2) above.
Adaptation parameter given by the difference of the deviance ratios

$$\frac{{SDX}_{m}}{{SDY}_{m}} - \frac{{SDX}_{n}}{{SDY}_{n}} = ADAP ,$$

for given m and n.

### Frequency-Domain Measures

These measures are typically computed by taking a frequency-domain transform, such as a Fourier transform, over the sequence of RR interval values, and then analyzing the resulting frequency spectrum.
Total spectral power of all RR intervals up to a certain frequency cutoff, typically 0.4 Hz (TOTPWR).
Total spectral power of all RR intervals in the ultra-low frequency range, typically up to 0.003 Hz (ULF).
Total spectral power of all RR intervals in the very low frequency range, typically 0.003-0.04 Hz (VLF).
Total spectral power of all RR intervals in the low frequency range, typically 0.04-0.15 Hz (LF).
Total spectral power of all RR intervals in the high frequency range, typically 0.15-0.4 Hz (HF).
Ratio of low- to high-frequency power components (LF/HF).

### Fractal Analysis Measures

These measures and their uses in analyzing HRV are described generally in the above-mentioned articles by Tan et al. and by Porta et al.
Self-similar fractal scaling (1/f).
Log transformation of the head of a detrended fluctuation analysis (DFA) graph (STT).
Log transformation of the tail of a detrended fluctuation analysis (DFA) graph (LTT).
Symbolic dynamics (SDyn).
Multiscale complexity measures.
   Computation of the fractal scaling f and DFA parameters, including STT and LTT, is further described by Goldberger et al., in “Fractal dynamics in Physiology: Alterations with Disease and Aging,” Proceedings of the National Academy of Sciences USA 99, pages 2466-72 (2002), and by Acharya et al., in “Heart Rate Analysis in Normal Subjects of Various Age Groups,” Biomedical Engineering Online 3:24 (2004). Both of these publications are incorporated herein by reference. Details of methods that may be used in the DFA, STT and LTT computations are presented in the Appendix below.

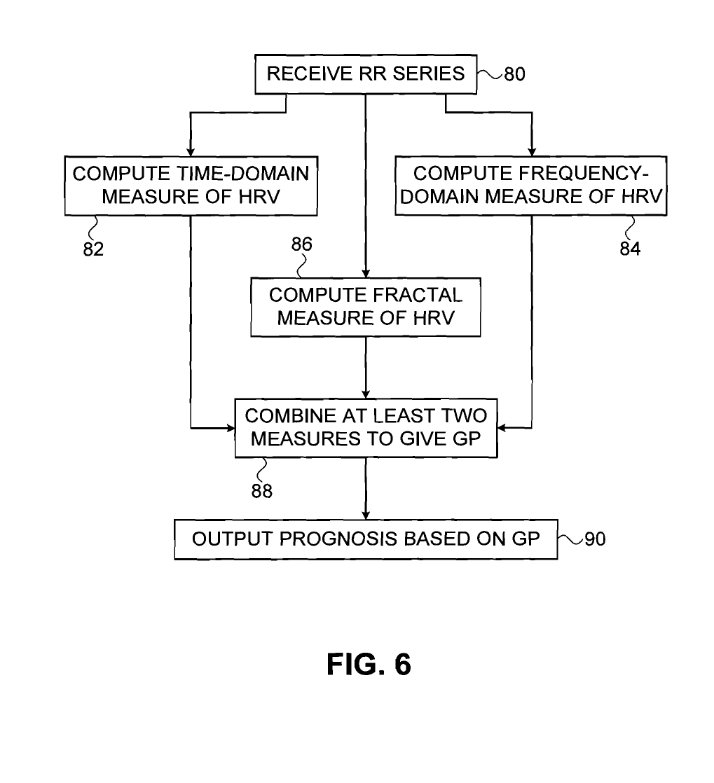

FIG. 6 is a flow chart that schematically illustrates a method applied by analysis module 32 in deriving a measure of cardiac health based on a generalized parameter, in accordance with an embodiment of the present invention. As in the method of FIG. 3, the analysis module applies the method to data comprising series of heartbeat intervals (typically RR intervals) that it receives from measurement module 24, at a data input step 80, and the data may be filtered to remove spurious data, as noted above.

Processor 36 computes at least two measures of HRV over the RR interval data, of at least two different types (among the three types listed above):
A time-domain measure 82;
A frequency-domain measure 84; and/or
A fractal analysis measure 86.
   The processor then combines these measures to derive the generalized parameter, at a combination step 88. The combination may be linear or non-linear, and may involved weighted sums, as well as raising certain measures to powers not equal to one, as illustrated in the examples below. Analysis module 32 compares the generalized parameter to an appropriate threshold, and then outputs a prognostic indicator depending on whether the parameters is above or below threshold, at an output step 90. Alternatively or additionally, the analysis module may apply other, more complex criteria in evaluating the generalized parameters calculated at step 88.

The generalized parameter GP that is calculated at step 88 typically has the general form:

GP=Σᵢaᵢ(Xᵢ)^{b}^{ᵢ} (3)

Here Xᵢ=Pᵢ−Cᵢ, wherein Pᵢ is the value of measure i, and Cᵢ is a cutoff value, which is determined empirically. The coefficient aᵢ=Sᵢ/Cᵢ, wherein Sᵢ represents a percentage of success, meaning the fraction of patients in the test sample for whom the parameter correctly indicated the patient's state of cardiac health. The power bᵢ is likewise determined empirically. The values of Sᵢ, Cᵢ and bᵢ may be chosen by computing the generalized parameter over a test set of known HRV data (such as the CAST dataset mentioned above), and then optimizing the values to maximize the separation of the healthy from the diseased cohort on the basis of the calculation. Any suitable method that is known in the art may be used to optimize these values, such as linear or logistic regression, support vector machines, neural networks, or trial-and-error search. The threshold for distinguishing between the healthy and diseased cohorts is likewise determined empirically so as to maximize the sensitivity and specificity of the GP over the test data.

The following tables present a number of generalized parameters that may be computed using the method of FIG. 6, with representative coefficients, powers, cutoff and success values:

TABLE II
GP BASED ON TIME DOMAIN AND FRACTAL ANALYSIS

| i | Pᵢ | Cᵢ | Sᵢ | aᵢ | bᵢ |
|---|---|---|---|---|---|
| 1 | SDANN | 200 | 0.60 | 0.003 | 1.3 |
| 2 | STT | 180 | 0.50 | 0.00278 | 1 |

TABLE III
GP BASED ON TWO TIME-DOMAIN MEASURES AND FRACTAL
ANALYSIS

| i | Pᵢ | Cᵢ | Sᵢ | aᵢ | bᵢ |
|---|---|---|---|---|---|
| 1 | RATIO | 4 | 0.80 | 0.2 | 1 |
| 2 | LTT | 250 | 0.70 | 0.0028 | 1 |
| 3 | ADAP | 1.6 | 0.85 | 0.53125 | 1 |

TABLE IV
GP BASED ON THREE TIME-DOMAIN MEASURES AND FRACTAL
ANALYSIS

| i | Pᵢ | Cᵢ | Sᵢ | aᵢ | bᵢ |
|---|---|---|---|---|---|
| 1 | RATIO | 4 | 0.80 | 0.2 | 1 |
| 2 | SDANN | 200 | 0.60 | 0.003 | 0.5 |
| 3 | STT | 180 | 0.50 | 0.00278 | −0.2 |
| 4 | ADAP | 1.6 | 0.85 | 0.53125 | 2 |

TABLE V
GP BASED ON THREE TIME-DOMAIN AND TWO FRACTAL
ANALYSIS MEASURES

| i | Pᵢ | Cᵢ | Sᵢ | aᵢ | bᵢ |
|---|---|---|---|---|---|
| 1 | RATIO | 4 | 0.80 | 0.2 | 1 |
| 2 | SDANN | 200 | 0.60 | 0.003 | 1.3 |
| 3 | STT | 180 | 0.50 | 0.00278 | 1 |
| 4 | LTT | 250 | 0.70 | 0.0028 | 1 |
| 5 | ADAP | 1.6 | 0.85 | 0.53125 | 1 |

TABLE VI
GP BASED ON TIME-DOMAIN AND FRACTAL ANALYSIS
MEASURES

| i | Pᵢ | Cᵢ | Sᵢ | aᵢ | bᵢ |
|---|---|---|---|---|---|
| 1 | LTT | 250 | 0.70 | 0.0028 | 1 |
| 2 | RATIO | 4 | 0.80 | 0.2 | 1 |
| 3 | ADAP | 1.6 | 0.85 | 0.53125 | 1 |

TABLE VII
COMPOUND GP COMBINING TIME-DOMAIN,
FREQUENCY-DOMAIN AND FRACTAL ANALYSIS MEASURES

| i | Pᵢ | Cᵢ | Sᵢ | aᵢ | bᵢ |
|---|---|---|---|---|---|
| 1 | GP (as defined in Table VI) | −0.374 | −0.061 | 0.162 | 1 |
| 2 | b (coefficient in equation 2) | −0.925 | 0.082 | −0.089 | 1 |
| 3 | LF/HF | 1.236 | 0.231 | 0.187 | 1 |

The formula in Table VII includes the results of lag-based time-domain analysis, which is performed using the method of FIG. 3, with other measures that are defined above.

Although certain specific formulas for calculation of generalized parameters are presented above as non-limiting examples by way of illustration, other formulas embodying similar principles will be apparent to those skilled in the art after reading the above description and are considered to be within the scope of the present invention. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

### APPENDIX—DETRENDED FLUCTUATION ANALYSIS (DFA)

Software that may be used in performing DFA of HRV is available on the PhysioNet Web site at www.physionet.org/physiotools/dfa/. As explained on this site, DFA reveals the extent of long-range correlations in time series. The heart rate time series to be analyzed comprises N samples, which are denoted HR(i). This series is integrated to give the time series

$$y ( k ) = {\sum }_{i = 1}^{k} [ HR ( i ) - {HR}_{avg} ]$$

and is then divided into boxes of equal length, n. In each box, a least-squares line is fitted to the data (representing the trend in that box). The y coordinate of each straight line segment is denoted yₙ(k). The integrated time series y(k) is detrended by subtracting the local trend yₙ(k) in each box. The root-mean-square fluctuation of this integrated and detrended time series is given by:

$$F ( n ) = \sqrt{\frac{1}{N} {\sum }_{k = 1}^{N} {[ y ( k ) - {y}_{n} ( k ) ]}^{2}}$$

This fluctuation computation is repeated over all time scales (different box sizes n), giving an array of pairs V=[(n₁,F(n₁)),(n₂,F(n₂)), . . . , (nₘ,F(nₘ))], which characterizes the relationship between the fluctuation and the box size. Typically, F(n) will increase with box size. When power law (fractal) scaling is present, the fluctuations can be characterized by a scaling exponent, given by the slope of the line relating log F(n) to log n.

The long-term trend (LTT) and short-term trend (STT) values are computed by applying a log transformation to a selected group of K elements of V:

$$DFA 2 = \frac{1}{K} {\sum }_{i = 0}^{K - 1} {log}_{{n}_{m - 1}} ( F ( {n}_{m - 1} ) )$$

To calculate LTT, DFA2 is calculated over the last K elements of V (wherein K is typically on the order of 10) and is then truncated and scaled as follows:

LTT=(round(DFA2,5)−1)*1000

Here the “round” function above rounds DFA2 to 5 places to the right of the decimal point. SIT is calculated in the same manner, except that only the first K elements of V are included in the DFA2 sum.

The specific formulas and parameters that are used above in computing the LTT and STT values are given here by way of example. Alternative methods for extracting trend values from fractional analysis will be apparent to those skilled in the art, and their use in computing generalized parameters for predicting cardiac health is considered to be within the scope of the present invention.

1. A diagnostic method, comprising:
receiving data comprising a series of heartbeat intervals acquired from a patient;
applying a first type of computation, selected from a group of computation types consisting of time-domain analysis, frequency-domain analysis, and nonlinear fractal analysis, to the data in order to compute a first measure of heart rate variability (HRV) of the patient;
applying a second type of computation, selected from the group and different from the first type, to the data in order to compute a second measure of the HRV of the patient; and
combining at least the first and second measures so as to derive a parameter indicative of a condition of cardiac health of the patient.

2. The method according to claim 1, wherein combining at least the first and second measures comprises computing a weighted sum of the first and second measures.

3. The method according to claim 1, wherein combining at least the first and second measures comprises computing a non-linear function of at least one of the first and second measures.

4. The method according to claim 3, wherein computing the non-linear function comprises raising at least one of the first and second measures to a power not equal to one.

5. The method according to claim 1, and comprising setting a threshold, and comparing the parameter to the threshold in order to provide a prognostic classification of the patient.

6. The method according to claim 1, wherein the time-domain analysis comprises computing a variation of the heartbeat intervals between pairs of the heartbeats as a function of a lag between the heartbeats in each pair, and deriving a measure from the function.

7. The method according to claim 1, wherein the time-domain analysis comprises computing a measure selected from a set of measures consisting of:
a mean RR interval;
a standard deviation of RR intervals;
a ratio of standard deviations along different axes in a scatter plot of the RR intervals;
a difference between ratios of standard deviations along different axes in scatter plots of the RR intervals computed for different lags between heartbeats;
a standard deviation of an average of RR intervals in different time segments;
a mean square difference between adjacent RR intervals; and
a fraction of differences between adjacent RR intervals that are greater than a certain time threshold.

8. The method according to claim 1, wherein the frequency-domain analysis comprises computing a measure selected from a set of measures consisting of:
a total spectral power of all RR intervals up to a frequency cutoff;
a total spectral power of all RR intervals in a specified frequency range; and
a ratio of power components of the RR intervals in two different frequency ranges.

9. The method according to claim 1, wherein the nonlinear fractal analysis comprises computing a measure selected from a set of measures consisting of:
a self-similar fractal scaling;
a log transformation of a head of a detrended fluctuation analysis (DFA) graph; and
a log transformation of a tail of a detrended fluctuation analysis (DFA) graph.

10. A diagnostic method, comprising:
receiving data comprising a series of heartbeat intervals acquired from a patient;
computing a variation of the heartbeat intervals between pairs of the heartbeats as a function of a lag between the heartbeats in each pair; and
analyzing the computed variation in order to assess a condition of cardiac health of the patient.

11. The method according to claim 10, wherein computing the variation comprises finding a ratio of first and second deviances along respective first and second axes in a scatter plot of the pairs.

12. The method according to claim 10, wherein analyzing the computed variation comprises fitting a parametric curve to the function, and comparing at least one parameter of the curve to a predefined criterion in order to assess the condition.

13. The method according to claim 12, wherein fitting the parametric curve comprises fitting a quadratic logarithmic function, and wherein comparing the at least one parameter comprises checking a sign of a parameter that multiplies a linear term in the function in order to assess the cardiac health.

14. Diagnostic apparatus, comprising:
a memory, which is configured to receive data comprising a series of heartbeat intervals acquired from a patient; and
a processor, which is configured to apply a first type of computation, selected from a group of computation types consisting of time-domain analysis, frequency-domain analysis, and nonlinear fractal analysis, to the data in order to compute a first measure of heart rate variability (HRV) of the patient, to apply a second type of computation, selected from the group and different from the first type, to the data in order to compute a second measure of the HRV of the patient, and to combine at least the first and second measures so as to derive a parameter indicative of a condition of cardiac health of the patient.

15. The apparatus according to claim 14, wherein the parameter comprises a weighted sum of the first and second measures.

16. The apparatus according to claim 14, wherein the parameter is derived by computing a non-linear function of at least one of the first and second measures.

17. The apparatus according to claim 16, wherein the non-linear function comprises raising at least one of the first and second measures to a power not equal to one.

18. The apparatus according to claim 14, wherein the processor is configured to compare the parameter to a predetermined threshold in order to provide a prognostic classification of the patient.

19. The apparatus according to claim 14, wherein the time-domain analysis comprises computing a variation of the heartbeat intervals between pairs of the heartbeats as a function of a lag between the heartbeats in each pair, and deriving a measure from the function.

20. The apparatus according to claim 14, wherein the time-domain analysis comprises computing a measure selected from a set of measures consisting of:
a mean RR interval;
a standard deviation of RR intervals;
a ratio of standard deviations along different axes in a scatter plot of the RR intervals;
a difference between ratios of standard deviations along different axes in scatter plots of the RR intervals computed for different lags between heartbeats;
a standard deviation of an average of RR intervals in different time segments;
a mean square difference between adjacent RR intervals; and
a fraction of differences between adjacent RR intervals that are greater than a certain time threshold.

21. The apparatus according to claim 14, wherein the frequency-domain analysis comprises computing a measure selected from a set of measures consisting of:
a total spectral power of all RR intervals up to a frequency cutoff;
a total spectral power of all RR intervals in a specified frequency range; and
a ratio of power components of the RR intervals in two different frequency ranges.

22. The apparatus according to claim 14, wherein the nonlinear fractal analysis comprises computing a measure selected from a set of measures consisting of:
a self-similar fractal scaling;
a log transformation of a head of a detrended fluctuation analysis (DFA) graph; and
a log transformation of a tail of a detrended fluctuation analysis (DFA) graph.

23. Diagnostic apparatus, comprising:
a memory, which is configured to receive data comprising a series of heartbeat intervals acquired from a patient; and
a processor, which is configured to compute a variation of the heartbeat intervals between pairs of the heartbeats as a function of a lag between the heartbeats in each pair, and to analyze the computed variation in order to assess a condition of cardiac health of the patient.

24. The apparatus according to claim 23, wherein the variation is represented by a ratio of first and second deviances along respective first and second axes in a scatter plot of the pairs.

25. The apparatus according to claim 23, wherein the processor is configured to fit a parametric curve to the function, and to compare at least one parameter of the curve to a predefined criterion in order to assess the condition.

26. The apparatus according to claim 25, wherein the parametric curve comprises a quadratic logarithmic function, and wherein the processor is configured to check a sign of a parameter that multiplies a linear term in the function in order to assess the cardiac health.

27. A computer software product, comprising a computer-readable medium in which program instructions are stored, which instructions, when read by a processor, cause the processor to receive data comprising a series of heartbeat intervals acquired from a patient, to apply a first type of computation, selected from a group of computation types consisting of time-domain analysis, frequency-domain analysis, and nonlinear fractal analysis, to the data in order to compute a first measure of heart rate variability (HRV) of the patient, to apply a second type of computation, selected from the group and different from the first type, to the data in order to compute a second measure of the HRV of the patient, and to combine at least the first and second measures so as to derive a parameter indicative of a condition of cardiac health of the patient.

28. The product according to claim 27, wherein the parameter comprises a weighted sum of the first and second measures.

29. The product according to claim 27, wherein the parameter is derived by computing a non-linear function of at least one of the first and second measures.

30. The product according to claim 29, wherein the non-linear function comprises raising at least one of the first and second measures to a power not equal to one.

31. The product according to claim 27, wherein the instructions cause the processor to compare the parameter to a predetermined threshold in order to provide a prognostic classification of the patient.

32. The product according to claim 27, wherein the time-domain analysis comprises computing a variation of the heartbeat intervals between pairs of the heartbeats as a function of a lag between the heartbeats in each pair, and deriving a measure from the function.

33. The product according to claim 27, wherein the time-domain analysis comprises computing a measure selected from a set of measures consisting of:
a mean RR interval;
a standard deviation of RR intervals;
a ratio of standard deviations along different axes in a scatter plot of the RR intervals;
a difference between ratios of standard deviations along different axes in scatter plots of the RR intervals computed for different lags between heartbeats;
a standard deviation of an average of RR intervals in different time segments;
a mean square difference between adjacent RR intervals; and
a fraction of differences between adjacent RR intervals that are greater than a certain time threshold.

34. The product according to claim 27, wherein the frequency-domain analysis comprises computing a measure selected from a set of measures consisting of:
a total spectral power of all RR intervals up to a frequency cutoff;
a total spectral power of all RR intervals in a specified frequency range; and
a ratio of power components of the RR intervals in two different frequency ranges.

35. The product according to claim 27, wherein the nonlinear fractal analysis comprises computing a measure selected from a set of measures consisting of:
a self-similar fractal scaling;
a log transformation of a head of a detrended fluctuation analysis (DFA) graph; and
a log transformation of a tail of a detrended fluctuation analysis (DFA) graph.

36. A computer software product, comprising a computer-readable medium in which program instructions are stored, which instructions, when read by a processor, cause the processor to receive data comprising a series of heartbeat intervals acquired from a patient, to compute a variation of the heartbeat intervals between pairs of the heartbeats as a function of a lag between the heartbeats in each pair, and to analyze the computed variation in order to assess a condition of cardiac health of the patient.

37. The product according to claim 36, wherein the variation is represented by a ratio of first and second deviances along respective first and second axes in a scatter plot of the pairs.

38. The product according to claim 36, wherein the instructions cause the processor to fit a parametric curve to the function, and to compare at least one parameter of the curve to a predefined criterion in order to assess the condition.

39. The product according to claim 38, wherein the parametric curve comprises a quadratic logarithmic function, and wherein the instructions cause the processor to check a sign of a parameter that multiplies a linear term in the function in order to assess the cardiac health.
